(19) United States
(12) Patent Application Publication
(10) Pub. No.: US 2009/0130677 A1
(43) Pub. Date: May 21, 2009

(54) METHOD FOR ISOTHERMAL AMPLIFICATION OF NUCLEIC ACIDS AND METHOD FOR DETECTING NUCLEIC ACIDS USING SIMULTANEOUS ISOTHERMAL AMPLIFICATION OF NUCLEIC ACIDS AND SIGNAL PROBE

(76) Inventors: Min Hwan Kim, Gyeonggi-Do (KR); Ji Won Jeong, Seoul (KR); Un Ok Kim, Gyeonggi-Do (KR)

Correspondence Address: IPLA P.A., 3580 WILSHIRE BLVD., 17TH FLOOR, LOS ANGELES, CA, 90010, US

(21) Appl. No.: 12/090,196

(22) PCT Filed: Sep. 8, 2006
(86) PCT No.: PCT/KR2006/003577

§ 371 Date: Apr. 14, 2008

(30) Foreign Application Priority Data
Oct. 14, 2005 (KR) 10-2005-0097003
Sep. 6, 2006 (KR) 10-2006-0085818
Sep. 8, 2006 (KR) PCT/KR2006/003577

(51) Int. Cl. C12Q 1/68; C12P 19/34

(52) U.S. Cl. 435/6; 435/91.2; 435/91.21

(57) ABSTRACT

The present invention relates to a method for isothermal amplification of nucleic acids and a method for detecting nucleic acids, which comprises characterized in simultaneous isothermal amplification of nucleic acids and a signal probe to a method for isothermal amplification of target nucleic acids using an external primer set and RNA/DNA hybrid primer set, and a method for detecting amplification products by amplifying nucleic acids and a signal probe simultaneously using an external primer set, RNA-DNA hybrid primer set and DNA-RNA-DNA hybrid probe. The method according to the present invention is convenient compared with the conventional method, it is possible to amplify the target nucleic acids rapidly and exactly without a risk of contamination, and it can simultaneously amplify a signal probe, so that it can be applied to various genome project, such as detection and identification of a pathogen, detection of gene modification causing predetermined phenotype, detection of hereditary diseases or determination of sensibility to diseases, estimation of gene expression and apply to genome project, thus being useful for molecular biological studies and disease diagnosis.

### TECHNICAL FIELD

The present invention relates to a method for isothermal amplification of nucleic acids and a method for detecting a nucleic acid, which comprises simultaneous isothermal amplification of nucleic acids and a signal probe for detecting amplification product, and more particularly to a method for isothermal amplification of target nucleic acids using external primer set and RNA/DNA hybrid primer set, and a method for detecting amplification product by simultaneously amplifying nucleic acids and signal probe using external primer set, RNA-DNA hybrid primer set and DNA-RNA-DNA hybrid probe.

### BACKGROUND ART

Nucleic acid amplification techniques are very useful in detecting and analyzing a small quantity of nucleic acid. A high sensibility of nucleic acid amplification to target nucleic acids enabled the development of the technology for detecting specific nucleic acids in terms of gene separation for diagnosis and analysis of infectious diseases and genetic diseases and medicolegal aspect. Based on such methods for detecting nucleic acids, various methods which can perform substantially sensitive diagnosis and analysis have been developed (Belkum, Current Opinion in Pharmacology, 3:497, 2003).

Detection of nucleic acid is caused due to complementarity of DNA strands and the ability to form hybrid molecules of double strands from single stranded nucleic acid in vitro. Due to this ability, it is possible to detect specific nucleic acids in a sample (Barry et al., Current Opinion in Biotechnology, 12:21, 2001).

Probe used in detection of nucleic acid is composed of specific sequences capable of hybridizing with a target sequence existed in nucleic acid sample. The probe is read by chemical materials, immuno-chemical materials, fluorescent materials or radioisotopes. Generally, the probe is constructed to include fluorescent materials capable of reading DNA hybridization and fragmentary nucleic acids having complementary sequence to target nucleic acids, or marker or reporter molecules such as biotin and digoxigenin.

However, the above method has problems in that it cannot detect a short sequence on the chromosomal DNA, has low copy numbers and has a difficulty to solve the problem of the limited copy number of modified allele of wild-type gene. Another problem of the method is related to environmental conditions of in vitro or in situ, which limit physical interaction among target sequence, chemical materials, probe and another molecular or structure.

The method for detection of target nucleic acid is classified into three categories, that is, (1) target sequence amplification which amplifies target nucleic acids, (2) probe amplification which a probe molecule itself, and (3) signal amplification signal shown by each probe by complex probe or a probe coupling method.

In vitro nucleic acid amplification techniques have been used as leading methods in detecting and analyzing a small quantity of nucleic acid. Among them, PCR (polymerase chain reaction) has been used most widely as a nucleic acid amplification technique, in which, and a copy of each strand of complementary sequence is synthesized as synthesis of nucleic acids by primer is progressed repeatedly using each strand of complementary sequence as template. In order to carry out PCR, pre-programmed thermal cycling instrument is needed. Therefore, PCR technique has the following shortcomings: it costs a lot; it has a relatively low specificity; it requires extreme performance standard to reperform the results.

In LCR (ligase chain reaction) which is another nucleic acid amplification technique, two neighboring oligonucleotides are hybridized with target nucleic acids, and then ligased with ligase. Probe formed through this method is amplified by temperature cycling together with complementary nucleotide.

Since LCR has higher discriminatory power than primer extension, it shows higher allele specificity than that of PCR in genotyping point mutation. Among nucleic acid amplification techniques developed up to now, LCR has the highest specificity and it is the easiest method to use because all of discrimination mechanisms are optimized. However, it has shortcoming in that its reaction rate is the slowest and it requires many modified probes.

In methods using ligation such as LCR, genotyping can be performed by amplifying with RCA technique using primarily circularized padlock probe through DNA ligation accompanied in process of LCR or RCA (rolling circle replication) without target amplification PCR (Qi et al., Nucleic Acids Res., 29:e116, 2001)

SDA (strand displacement amplification) is an amplification method of a target nucleic acid sequence and the complementary strand thereof in samples by strand displacement using endonuclease. This method uses a mixture containing nucleic acid polymerase, at least one complementary primer to 3′-terminal end of target fragment and dNTPs (deoxynucleoside triphosphate) comprising at least one substituted dNTP. Each primer has a sequence in 5′-terminal end, which restriction endonuclease can recognize (Walker et al., Nucleic Acids Res., 29:1691, 1992).

As similar methods to SDA, there are SPIA (single primer isothermal amplification) technique using 5′-RNA/DNA-3′ primer (U.S. Pat. No. 6,251,639), ICAN (isothermal chimeric primer-initiated amplification of nucleic acid) technique using 5′-DNA/RNA-3′ primer (US appl. 2005/0123950) and Riboprimer technique using RNA primer (US appl. 2004/0180361) etc, in which after an extension of primer using RNA/DNA hybrid primer or RNA primer, primer and template DNA is digested with RNaseH digesting RNA primer hybridized with template DNA, and then a new primer is extended by strand displacement.

TMA (transcription mediated amplification) is a target nucleic acid amplification technique using only one promoter-primer at constant temperature, constant ionic strength and constant pH (Kwoh et al, Proc. Nat. Acad. Sci. USA, 86:1173, 1989). TMA comprises the step of combining a mixture composed of target nucleic acids and promoter-primer which is a complementary oligonucleotide to 3′-terminal end of target sequence for hybridization with 3′ terminal of target nucleic acids or neighboring region thereof. The promoter-primer comprises a sequence of promoter region for RNA polymerase located in 5′-terminal end of complexing sequence. The promoter-primer and target sequence form primer/target sequence hybrid to extend DNA.

In the process of DNA extension of TMA technique, it is assumed that the 3′-terminal end of target sequence was extended from location close to complex in which promoter-primer is hybridized between complexing sequence and target sequence. Promoter sequence produces a first DNA extension product to act as a template in extension process forming double stranded promoter sequence. The 3′ terminal of promoter-primer could be used as a primer in the second extension process. In the extension process, double stranded nucleic acid complex is formed using target sequence as template. When a RNA target sequence is used, the complex is DNA/RNA complex and when a DNA target sequence is used, the complex is DNA/DNA complex. Subsequently, RNA polymerase recognizing a promoter of promoter-primer synthesizes RNA using the first DNA extension product in order to produce various RNA copies of target sequence.

NASBA (nucleic acid sequence-based amplification) technique comprises syntheses of single stranded RNA, single stranded DNA and double stranded DNA (Compton, Nature, 350:91, 1991). The single stranded RNA becomes the first template for the first primer, the single stranded DNA becomes the second template for the second primer, and the double stranded DNA becomes the third template in synthesis of copies for the first template.

There is a problem in that the amplification method using heat cycle process such as PCR requires a thermal heat block to reach “target” temperature of each cycle, and a delay time till the heat block reaches the target temperature, therefore it takes long time till the amplification reaction is finished.

Since the method for isothermal amplification of target nucleic acids such SDA, NASBA and TMA is performed at constant temperature, it dose not require a separate thermal cycling apparatus, and thus, it is easy to handle.

However, the isothermal amplification of target nucleic acids disclosed up to now have several disadvantages. The method according to SDA requires a specific region for a given restriction enzyme, so the application is limited. The transcription-based amplification methods such NASBA and TMA require the binding between polymerase promoter sequence and amplification product by primer, and this process tends to bring a non-specific amplification. Because of these disadvantages, the amplification mechanism of DNA target by transcription-based amplification method has not been established well.

Moreover, currently used amplification methods are disadvantageous in that the test sample has the possibility of being contaminated by the products of preceding amplification reaction, thereby causing a non-target specific amplification. In order to prevent this, the contamination detection methods of sample solution which employ various means and physical means for decontamination of the sample in the last step of amplification reaction or before the beginning of target nucleic acids amplification, has been studied, but most of them make amplification operation complicate.

As a method for amplifying probe which is another method for detecting nucleic acid, there is a LCR method used in the said nucleic acid amplification method.

As another method for detecting nucleic acid, there is a method for amplifying a signal, not target nucleic acid and probe. Among these methods, there is an amplification method using four sets of probes to capture target nucleic acid (Ross et al., J. Virol. Method., 101:159, 2002). The hybrid capture method using signal amplification has sensitiveness worth being compared with methods for directly detecting and amplifying target nucleic acid, and uses an antibody and chemical luminous material for signal detection (Van Der Pol et al., J. Clinical Microbiol., 40:3564, 2002; Nelson et al., Nucleic Acids Research, 24:4998, 1996).

Furthermore, there is CPT (cycling probe technology) method as a method for amplifying signal probe (Duck et al., BioTechniques, 9:142, 1990). The method uses a DNA/RNA/DNA hybrid probe having the complementary base sequence to target nucleic acid, when the probe is hybridized with target nucleic acid, the RNA region of hybrid probe is digested with RNaseH, to be separated from the target nucleic acid, and then another DNA/RNA/DNA hybrid probe is hybridized with the target nucleic acid again to generate digested probe, thereby amplifying signal probe circularly.

However, the CPT method has disadvantages in that its amplification efficiency is 10³˜10⁶, which is relatively low, so it is difficult to use in diagnosis independently, the use thereof is complacate, since the signal probe is separately amplified after the amount of special region of target nucleic acid increased by conventional nucleic acid amplification such as PCR and it requires high costs and long time.

Accordingly, the present inventors made an extensive effort in order to overcome the problems described above and develop a method for amplifying target nucleic acids in rapid and exact manner, and at the same time, a method for detecting the amplification product, as a result, found that when external primer set having complementary base sequence to the target nucleic acids and RNA/DNA hybrid inner primer set having partially complementary base sequence to the target nucleic acid are used, it is possible to amplify the target nucleic acid rapidly at isothermal temperature. Moreover, they also confirmed that if DNA/RNA/DNA hybrid probe having a base sequence complementary to amplification product produced by the above method is used, it is possible to simultaneously amplify target nucleic acids and probe signals at isothermal temperature, thereby completing perfected the present invention.

### SUMMARY OF THE INVENTION

The object of the present invention is to provide a method for amplifying target nucleic acids at isothermal temperature rapidly and exactly.

Another object of the present invention is to provide a method for detecting nucleic acid, which comprises performing simultaneous isothermal amplification of nucleic acids and probe signal.

To achieve the above objects, the present invention provides an isothermal amplification method of nucleic acids, the method comprising the steps of: (a) denaturing a reaction mixture containing (i) target nucleic acids, (ii) external primer set having the complementary base sequence to the target nucleic acids, and (iii) RNA/DNA hybrid inner primer set having partially complementary base sequence to the target nucleic acids; and (b) adding an enzymatic reaction mixture solution containing RNase and DNA polymerase capable of strand displacement to the reaction mixture denatured in step (a), to amplify said target nucleic acids at isothermal temperature.

The present invention also provides a method for detecting nucleic acids, the method comprising the steps of: (a) denaturing a reaction mixture containing (i) a nucleic acid sample for detecting target nucleic acid, (ii) an external primer set having complementary base sequence to the target nucleic acids, and (iii) RNA/DNA hybrid inner primer set having partially complementary base sequence to target nucleic acid; (b) adding an enzymatic reaction mixture solution containing RNase and DNA polymerase capable of strand displacement, and DNA/RNA/DNA hybrid probe having complementary base sequence to amplification products produced by the external primer and interior primer sets to the reaction mixture denatured in step (a) to simultaneously amplify said target nucleic acids and said probe signals at isothermal temperature; and (c) detecting target nucleic acids using the amplified probe signals.

In the present invention, the external primer is preferably any one selected from the group consisting of: oligoDNA, oligoRNA and hybrid oligoRNA/DNA; and in RNA/DNA hybrid inner primer, RNA region thereof preferably has non-complementary base sequence to target nucleic acids and DNA region thereof preferably has complementary base sequence to target nucleic acids.

In the present invention, the DNA/RNA/DNA hybrid probe preferably consists of 25˜45 bases, the length of external DNA preferably consists of 10˜20 bases and the length of inner RNA consists of 4˜6 bases respectively.

In the present invention, the end of DNA/RNA/DNA hybrid probe is preferably labelled with marker, wherein the marker (label) is preferably materials binding selectively a the specific antibody such as biotin, fluorescent, dioxygenin and 2,4-dinitrophenyl and the like.

In the present invention, the 3′ end of DNA/RNA/DNA hybrid probe is preferably labelled with phosphate material to prevent probe extension during amplification process.

In the present invention, the DNA polymerase is preferably thermostable DNA polymerase, and the thermostable DNA polymerase is any one selected from the group consisting of: Bst DNA polymerase, exo(−) vent DNA polymerase, and Bca DNA polymerase.

In the present invention, the RNase is preferably RNaseH. In the present invention, the amplification of the target nucleic acids and the probe signals is preferably carried out at 50˜65° C.

Another features and embodiments of the present invention will be more clarified from the following “detailed description” and the appended “claims”.

### BRIEF DESCRIPTION OF DRAWINGS

FIG. 1 is a schematic figure of the method for isothermal amplification of nucleic acids according to the present invention.

FIG. 2 is a schematic figure of sequential form of isothermal amplification of nucleic acids according to the present invention.

FIG. 3 is a schematic figure of simultaneous amplification of nucleic acids and the signal probe according to the present invention.

FIG. 4 is an electrophoresis photograph of amplification products produced by the method for isothermal amplification of nucleic acids according to the present invention (M: marker, lane 1: sample without adding lambda DNA; lane 2: sample without adding inner and external primers; lane 3: sample without adding inner primer; lane 4: sample without adding external primer; lane 5: sample with an addition of lambda DNA, inner and external primers.

FIG. 5 is a schematic figure of detecting signal probe produced by amplification method according to the present invention, by means of aggregation of gold nanoprobe.

FIG. 6 is an analysis result of detecting signal probe produced by amplification method according to the present invention, by means of aggregation of gold nanoprobe.

FIG. 7 is a schematic figure of detecting signal probe produced by amplification method according to the present invention, by means of HRP (horse radish peroxidase)

FIG. 8 is an analysis result of detecting signal probe produced by amplification method according to the present invention, by means of HRP (horse radish peroxidase)

### DETAILED DESCRIPTION OF THE INVENTION, AND PREFERRED EMBODIMENTS

The present invention, in one aspect, is related to a method for isothermal amplification of nucleic acids.

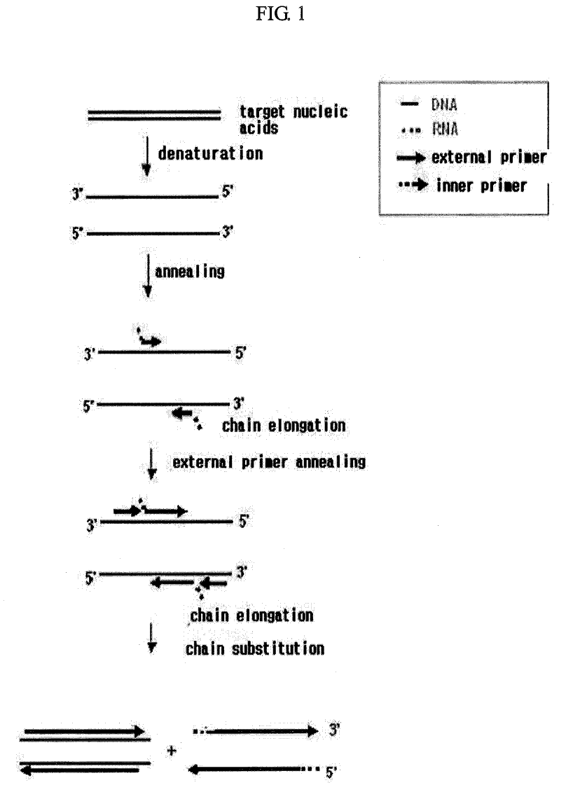

The isothermal amplification of nucleic acids according to the present invention is carried out with the following processes as showed in FIG. 1.

A mixture of target nucleic acids to be amplified as a template in amplification, external primer set and RNA/DNA hybrid inner primer set is first denatured to render each of them single stranded. The denatured mixture is cooled to isothermal amplification temperature, and an enzymatic reaction mixture solution containing RNase and DNA polymerase is added thereto. The external primer set and RNA/DNA hybrid inner primer set are then annealed with the target nucleic acids in reaction solution cooled to amplification temperature.

Preferably, the external primer set comprises a complementary sequence to a sequence closer to both ends of the target nucleic acids than the inner primer set, and the inner primer set comprises a sequence closer to the middle of the target nucleic acids than the external primer set. In this case, the inner primer is annealed in the forward direction of DNA strand extension compared with the external primer. The annealed external primer and inner primer are extended using a DNA polymerase capable of strand displacement. As the external primer is extended along template (target nucleic acids), the inner primer located in the forward direction of extension and DNA strand extended from the inner primer are separated from template (target nucleic acids) to result in a strand displacement. Finally, the amplification products of single stranded DNA, extended by inner primer and external primer, respectively, are obtained.

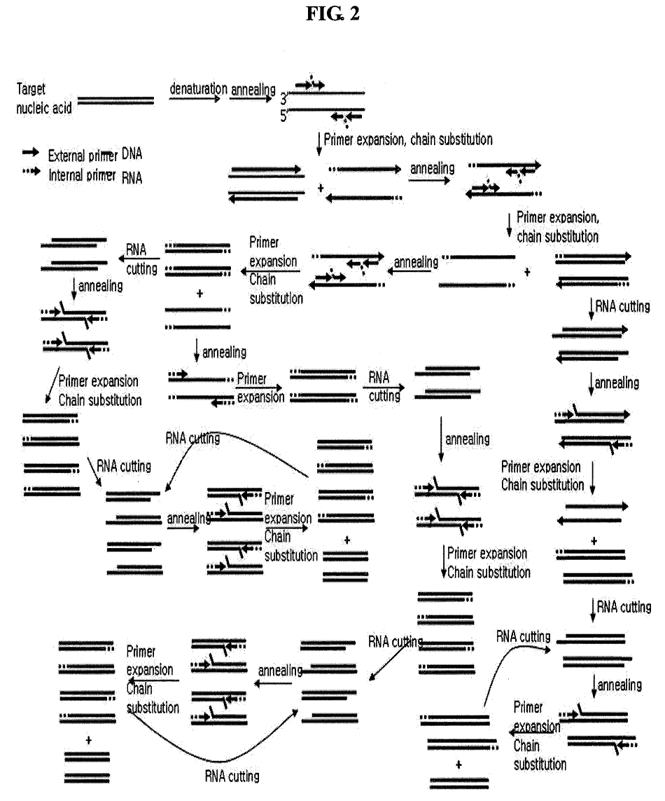

The external primer is extended using single stranded DNA as template to form double stranded DNA, the extended RNA/DNA hybrid inner primer is separated by strand displacement to obtain single stranded DNA, the RNA region of the double stranded DNA also is separated by RNaseH, and the RNA/DNA hybrid inner primer is annealed and then extended by strand displacement. The above described process is repeated to amplify the target DNA (FIG. 2).

The present invention, in another aspect, is a method for detecting nucleic acids, the method comprising simultaneously amplifying the nucleic acids and a signal probe for detecting amplification product.

A mixture of a nucleic acid sample for detecting target nucleic acids, an external primer set and RNA/DNA hybrid inner primer set is first denatured to make a single stranded DNA respectively. The denatured mixture is cooled to isothermal temperature, and added with enzymatic reaction mixture solution containing DNA polymerase, RNase, and DNA/RNA/DNA hybrid probe. Then, the external primer set and RNA/DNA hybrid inner primer set are annealed with the target nucleic acids in the reaction solution cooled to amplification temperature.

Preferably, the external primer set comprises complementary sequence to a sequence closer to both ends of the target nucleic acids, and the inner primer set comprises a sequence closer to the middle of the target nucleic acids. In this case, the inner primer is annealed in the forward direction of DNA strand extension compared with the external primer. The annealed external primer and inner primer are extended using a DNA polymerase capable of strand displacement, and as the external primer is extended along template (target nucleic acids), the inner primer located in the forward direction of extension and DNA strand extended from the inner primer are separated from template (target nucleic acids) to result in a strand displacement. Finally, the amplification products of single stranded DNA, extended by inner primer and external primer, respectively, are obtained.

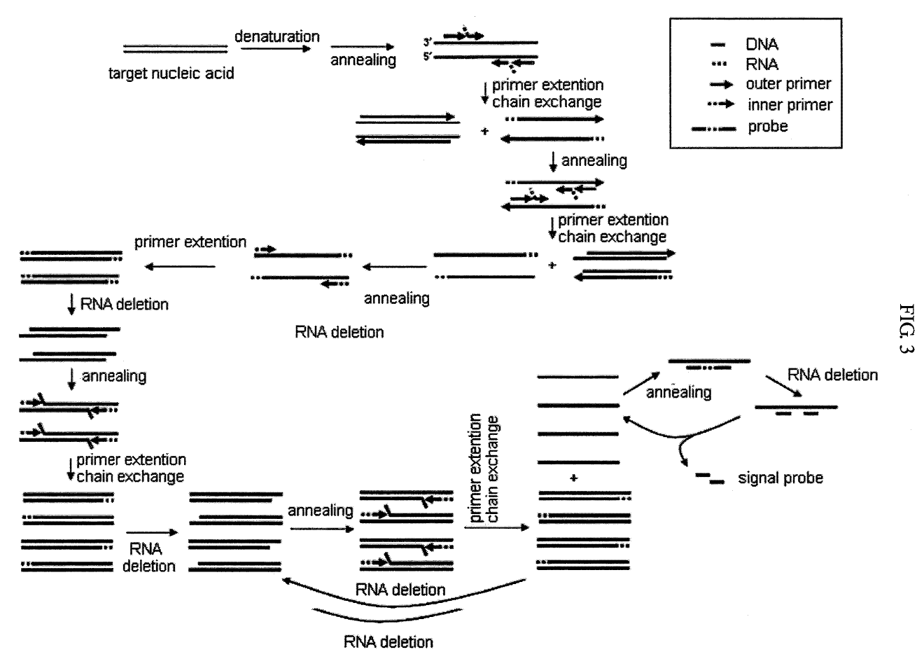

According to the present invention, the amplification of the probe signals is simultaneously performed with isothermal amplification of nucleic acids. After the target DNA amplified with isothermal amplification of nucleic acids is annealed with DNA/RNA/DNA hybrid probe to form RNA/DNA hybrid double strand, the RNA region of DNA/RNA/DNA hybrid probe is digested by RNaseH activity. Then, the signals of the RNA-digested probe are activated to separate the probe from the target DNA, followed by the binding of a new DNA/RNA/DNA hybrid probe to be digested with RNaseH and separated. The above described cycle is repeated to amplify the probe signals (FIG. 3).

In the present invention, the external primer is complementary to the sequence of the target nucleic acids, and preferably has 15˜30 bases, a target nucleic acids sequence complementary to an external primer is preferably a neighboring sequence of a target nucleic acids sequence complementary to an inner primer (the base difference is 1˜60 bp) and a target nucleic acids sequence complementary to an external primer is preferably sequence closer to 3′ end of the target nucleic acids than a nucleic acids sequence complementary to an inner primer.

In the present invention, the RNA/DNA hybrid inner primer is a primer in which oligoRNA is bound to oligoDNA, it is preferable that its 5′ end is non-complementary to the base sequence of target nucleic acids, and its 3′ end is complementary to the base sequence of target nucleic acids. Preferably, RNA/DNA hybrid inner primer consists of 20˜45 bases, wherein oligo RNA is 15˜25 bases and oligoDNA is 5˜20 bases.

More preferably, the oligoRNA in RNA/DNA hybrid inner primer is non-complementary to the base sequence of target nucleic acids and the oligoDNA is complementary to the base sequence of target nucleic acids.

In the present invention, the sequence of target nucleic acids complementary to RNA/DNA hybrid inner primer preferably comprises a sequence located closer to 5′ end than a sequence of target nucleic acids complementary to the external primer, and the sequence of target nucleic acids complementary to inner primer is preferably a neighboring sequence of the target nucleic acids sequence complementary to the external primer (the base difference is 1˜60 bp).

It is preferable that the DNA/RNA/DNA hybrid probe used in the present invention is oligonucleotide having a sequence complementary to nucleic acid amplification products amplified by the external primer or RNA/DNA hybrid inner primer, and the 5′ end and 3′ end of DNA/RNA/DNA hybrid probe consist of oligoDNAs and the middle thereof consists of oligoRNAs.

Preferably, the DNA/RNA/DNA hybrid probe consists of 25˜45 bases, the oligo DNA regions of 5′ end and 3′ end consist of 10˜20 bases respectively, and oligoRNA located at the middle consists of 4˜6 bases.

The signal probe amplified according to the present invention can be detected through the absorbance change in solution using cross-linking agglutination by hybridization of gold nano-oligo probes (Mirkin et al., Nature, 382:607-609, 1996). In this case, a probe having 3′ end tagged is preferably used in order to prevent from the DNA/RNA/DNA hybrid probe from being extended by DNA polymerase, and more preferably, a probe tagged with phosphate is used.

The gold nano-oligo probes whose 3′ end and 5′ end are conjugated to gold nano particles show a maximum absorbance at 530 nm in aqueous solution, but when a signal probe having a sequence complementary to the gold nano-oligo probe is present, the gold nano probe is cross-linked by hybridization to induce agglutination of gold nano probe, thus causing the maximum absorbance change. The existence of probe could be confirmed by measuring the change ratio of absorbance at 530 nm and 700 nm.

The signal probe amplified according to the method of the present invention can be detected using horse radish peroxidase in microplate (Bekkaoui et al., Diagn. Microbiol. Infect. Dis., 34:83-93, 1999). The end of DNA/RNA/DNA hybrid probe is preferably labelled with fluorescent material and biotin. After binding the probe with fluorescent material and biotin using microwell surface-treated with streptavidine binding with biotin selectively and HRP (horse radish peroxidase) conjugated with anti-fluorescent material binding with fluorescent material selectively, the resulting mixture is washed, and then TMB (tetranitrobenzidine, which is a substrate of HRP) reaction is performed to measure the change ratio of absorbance at 465 nm, thereby making it possible to confirm the existence of the probe, but it is not limited thereto. In addition to fluorescent material and biotin, a marker, conjugated with antibody binding selectively to the above described materials using 2,4-dinitrophenyl or digoxigenin, can be also used.

The DNA polymerase used in the present invention is an enzyme that can extend nucleic acid primer along DNA template, and should be capable of substituting for nucleic acid strand from polynucleotide bonding with substituted strand. DNA polymerase that can be used in the present invention is preferably Bst DNA polymerase, Bca DNA polymerase, exo(−) vent DNA polymerase, exo(−) Deep vent DNA polymerase, exo(−) Pfu DNA polymerase or pi29 DNA polymerase etc., which is known to be capable of strand displacement, and a thermostable DNA polymerase is preferable for the quickness and efficiency of the reaction.

The RNase used in the present invention generally digests the 5′RNA region and it is specific to digest the RNA strand of RNA/DNA hybrid, and it is preferable not to degrade a single strand RNA, and to use RNaseH.

In the present invention, the amplification reaction is preferably performed at the temperature at which the inventive primer and template DNA can be annealed and the activity of used enzyme is not substantially inhibited. The temperature, at which the amplification is performed, is preferably 30˜75° C., more preferably 37˜70° C., most preferably 50˜65° C.

The inventive thermal amplification of nucleic acids has high specificity, since it uses an additional external primer compared with conventional methods where a single RNA/DNA hybrid primer is used (U.S. Pat. No. 6,251,639). Besides, it is possible to significantly improve amplification efficiency by exponentially amplifying due to the inner primer substituted for the external primer which acts as a new template.

Moreover, the conventional method uses separate blocker blocking amplification or template switch oligonucleotide (TSO) to amplify a specific region upon the amplification of target base sequences using a single RNA/DNA hybrid primer on the contrary, the inventive method has an advantage in that only the desired region can be amplified using a pair of forward primer and reverse primer without using a separate blocker or TSO.

In still another aspect, the inventive method has an advantage in that it can simultaneously perform the amplification and detection of nucleic acids since the amplification of nucleic acids and a signal probe can be simultaneously completed in a single-tube by repeating a cycle where DNA/RNA/DNA hybrid probe is bound and separated using an amplified DNA as a template to amplify the signal probe.

In yet another aspect, the inventive method has an advantage in that it does not need to consider the problems occurring in the conventional method when the reaction activity of RNase is higher than primer extension activity of DNA polymerase, because the RNA region of RNA/DNA hybrid inner primer, used in the present invention, has a sequence non-complementary to a template.

In another further aspect of the inventive isothermal amplification of nucleic acids, when newly synthesized amplification product is used as a new template after a first primer extension and strand displacement reaction, the RNA region non-complementary to the template acts as template complementary to a primer to raise the annealing temperature with the primer, thus increasing amplification efficiency, as well as, preventing primer-dimer formation to enhance purity of amplification product.

The inventive isothermal amplification method and detection method of nucleic acids can be amplify in a rapid and simple manner since it employs one-step method in which the reaction is carried out at constant temperature, and it does not require a separate heat transducer, due to isothermal amplification of target nucleic acids and a signal probe. Additionally, the method exactly amplifies only target nucleic acids by using two pairs of primers and probes compared with conventional method, as well as, amplifies signal probes, thereby having excellent specificity.

In still another further aspect, the inventive isothermal amplification method and detection method of nucleic acids is carried out in one-tube and thus it is possible to treat in large quantities for real-time detection of nucleic acids. Such advantage can minimize the risk of an additional reaction by contamination, which limits a wide use of amplification technique.

The method for isothermal amplification of nucleic acids according to the present invention requires about 1 hr until complete amplification, starting from DNA extraction in sample, if DNA extraction was already completed, it requires about 40 min, thereby resulting in an advantage of performing rapid amplification reaction.

### EXAMPLES

Hereinafter, the present invention will be described in more detail by examples. However, it is obvious to a person skilled in the art that these examples are for illustrative purpose only and are not construed to limit the scope of the present invention.

### Example 1

### Isothermal Amplification of Nucleic Acids

Lambda DNA (TaKaRa Bio Inc.; 3010, 0.3 μg/ml) was used as target nucleic acids, an external primer and RNA/DNA hybrid inner primer were prepared by referring to whole base sequences of lambda DNA (GenBank No. J02459) and the conventional methods (Biochem. Biophy. Res. Comm., 289:150, 2001).

The external primer was designed such that it comprises sequences complementary to the lambda DNA, and the sequences are SEQ ID NOs: 1 or 2 as follows:

| | |
|---|---|
| SEQ ID NO: 1: | 5′-GGACGTCAGAAAACCAGAA-3′ |
| SEQ ID NO: 2: | 5′-GGCAGTGAAGCCCAGAT-3′ |

RNA/DNA hybrid inner primer was designed such that oligoRNA region thereof has a sequence non-complementary to lambda DNA, and oligoDNA region thereof has a sequence complementary to lambda DNA, and the sequences are SEQ ID NOs: 3 and 4 as follows (the oligoRNA regions are underlined):

SEQ ID NO: 3:
5′-UAAGAGAUCGCCCGUCAGCCGCTCCGATCACCCTCGCAAAC-3′
SEQ ID NO: 4:
5′-CAACAUGACCGACGCUUGCCCGCGCCACGCTCCTTAATCTG-3′

In order to amplify target nucleic acids using the external primer set and RNA/DNA hybrid inner primer set, a reaction mixture containing the external primer set, inner primer set and target nucleic acids was prepared. 10 mM of (NH₄)₂SO₄, 10 mM of MgSO₄, 4 mM of KCl, 0.5 mM of each dNTP (Fermentas), 0.5 mM of DTT, 0.1 μg of BSA, 0.1 μM of external primer set, 0.5 μM of inner primer set and 10 ng of lambda DNA were added to 20 mM of Tris-Hcl (pH 8.5) buffer to prepare the reaction mixture. The reaction mixture was denatured for 5 min at 95° C., cooled for 5 min at 63° C., and added with an enzymatic reaction mixture solution to the final volume of 20 μl for DNA amplification, followed by carrying out isothermal amplification for 1 hr at 63° C.

The components of enzymatic reaction mixture solution are as follows: 0.3 μg of T4 Gene 32 protein (USB), 6 units of RNase inhibitor (Inrton), 0.5 unit of RNaseH (Epicentre) and 30 units of Bst DNA polymerase (NEBM0275M).

On the other hand, as a control, an enzymatic reaction mixture solution without target nucleic acids (lambda DNA) and a reaction mixture without the external primer and/or the inner primer were used.

The reaction solution 6 μl taken after the amplification reaction was mixed with loading buffer, and subjected to electrophoresis on 1.8% agarose gel containing ethidium bromide, followed by determining amplification efficiency with a band on the in UV transilluminator.

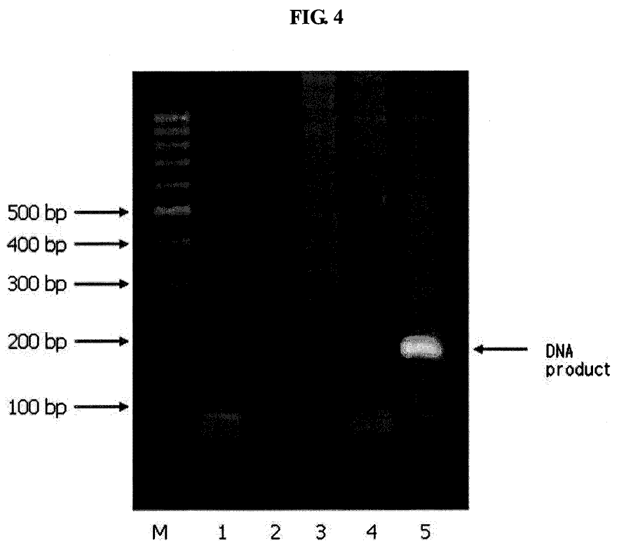

As a result, as showed in FIG. 4, it was confirmed that the amplification products of target nucleic acids were increased remarkably in the sample added with lambda DNA, the external primer and the inner primer compared with the sample without adding lambda DNA and the sample without adding the inner primer and/or the external primer.

### Example 2

### Isolation of Nucleic Acids from E. coli 0157:H7

E. coli 0157:H7 (KCCM 40406) was cultured in a suitable medium (Trypticase soy broth) at 37° C. under aerobic condition, from which cell culture broth was taken every hour to measure the absorbance and cell number. After carrying out a technique of diluting cell culture broth continuously in a liquid medium, cell number was enumerated according to absorbance using Helber cell counting chamber.

Isolation of nucleic acids in E. coli 0157:H7 was performed using G-spin Genomic DNA isolation system of Intron Inc (Cat NO: 17121). The isolation is as follows: 1.0 ml of bacteria culture broth at the logarithmic phase was taken to adjust to a concentration of 10⁷ cells/ml, the cells obtained by centrifuging at 13,000 g for 2 min was added with 300 ml of G-buffer solution to resuspend, and then left to stand for 15 min at 65° C., to which 250 ml of binding buffer was added to suspend smoothly. The binding buffer contains RNase solution and Proteinase K solution. The resulting mixture solution was moved to a spin column, and centrifuged at 13,000 g for 1 min. 500 ml of washing buffer A was added to the spin column and centrifuged at 13,000 rpm for 1 min to wash. Then, 500 ml of washing buffer B was added to spin column and centrifuged at 13,000 g for 1 min. The column was moved to a new tube, and column membrane was soaked evenly in 100 ml of elution buffer, and then left to stand for 3 min at room temperature, followed by centrifuging at 13,000 g for 2 min, thus obtaining nucleic acids. The obtained nucleic acids were stored at −80° C. to use.

### Example 3

### Preparation of Gold Nanocolloid [Diameter: 42 nm]

Synthesis of gold nanoparticle is performed using the conventional method known in the art (Liu et al., J. Am. Chem. Soc., 126:12299, 2004). The method is as follows: 200 ml of 0.3 mM HAuCl₄ (Aldrich, USA) was added into a flask and heated to stir, and then 1.8 ml of 38.8 mM sodium citrate (Aldrich, USA) was dropwised rapidly. The reaction mixture whose color changed from pale yellow to deep purple, was heated for 10 min to stir for 15 min while cooling it to room temperature when it became pale red, thus obtaining gold nanoparticle solution with a diameter of 42 nm showing a maximum UV absorbance at 520 nm.

### Example 4

### Preparation of Signal Probe (3′- and 5′-Alkanethiol Oligonucleotide Modified Au Nanoparticle)

A signal probe was prepared by referring to the conventional method known in the art (Nucleic Acids Res., 33:e168, 2005; J. Biotechnol., 119:111, 2005). Alkanethiol oligonucleotides (IDT) of SEQ ID NOs: 5 and 6 were added to 42 nm of gold nanoparticles previously prepared in Example 3 to a concentration of 3 mM, respectively, and wrapped in foil and then left to stand for 16 hrs at room temperature. 0.1M of phosphoric acid (KH₂PO₄/K₂HPO₄) with pH 7.0 was added to the gold nanoparticle solution to make 10 mM phosphoric acid solution, to which 2M NaCl was added to make 0.05M Nacl, followed by finally preparing 0.3M NaCl after 24 hours. Salt solution was dropwised into the solution slowly. After 24 hours, supernatant was removed by centrifuging at 8,000 rpm for 15 min in order to eliminate an excessive amount of thiol-DNA. The resulting mixture was washed with 10 mM phosphoric acid (pH 7.0) and 0.1 M NaCl twice, and finally dissolved in the same 10 mM phosphoric acid (pH 7.0) and 0.1M NaCl to obtain a probe labelled with gold nanoparticles.

| | |
|---|---|
| SEQ ID NO: 5: | 5′-HS-(CH₂)₆-AGTGACAAAACGCAG-3′ |
| SEQ ID NO: 6: | 5′-AACTGCTCTGGATGC-(CH₂)₃-SH-3′ |

### Example 5

### Amplification of Target Nucleic Acids and a Signal Probe for Detecting Aggregation of Gold Nanoprobe

By referring to the whole base sequences of E. coli 0157:H7(KCCM 40406), primers (SEQ ID NOs: 7˜10) and a probe (SEQ ID NO: 11) were prepared for selectively amplifying a specific gene region (stx-2).

| | |
|---|---|
| SEQ ID NO: 7: | 5′-CGTTCCGGAATGCAAATC-3′ |
| SEQ ID NO: 8: | 5′-CATCGTATACACAGGAGC-3′ |
| SEQ ID NO: 9: | 5′-TACCTTAAGGCATGGGCTGACTACTACTCACTGGTTTCATCATATCT-3′ |
| SEQ ID NO: 10: | 5′-CTGCTACGCGTGTGAAGATGACCCTGAACAGTGCCTGACGAAATTCT-3′ |
| SEQ ID NO: 11: | 5′-TGCTGTGACAGTGACAAAACGCAGAACTGCTCTG-Phasphate-3′ |

The primers of SEQ ID NOs: 7 and 8 are external primers having a sequence complementary to Escherichia coli 0157:H7, and the primers of SEQ ID NOs: 9 and 10 are inner primers having a sequence complementary and a sequence non-specific to Escherichia coli 0157:H7. SEQ ID NO: 11 is a probe for performing an amplification of a signal probe, which is DNA/RNA/DNA hybrid probe tagged with phosphoric acid group at 3′ end thereof in order to prevent DNA extension reaction. In the above sequences, the underlined parts are RNA base sequences.

In order to anneal the primers with desired nucleic acids, a reaction mixture containing the external primers (SEQ ID NOs: 7 and 8), inner primers (SEQ ID NOs: 9 and 10) was denatured for 5 min at 95° C., and cooled for 5 min at 63° C.

The reaction mixture consists of the following constituents: 5 mM Tris-Hcl (pH 8.5), 10 mM of (NH₄)₂SO₄, 10 mM of MgSO₄, 4 mM of KCl, 0.5 mM of each dNTP (Fermentas), 2.9 mM of DTT, 0.1 mg of BSA, 10 mM of EGTA, 50 mM spermine, 0.08 mM of external primer, 0.126 mM of inner primer and 1 ng of E. coli 0157:H7 DNA.

Then, after adding enzymatic reaction mixture solution to the cooled reaction mixture to a final volume of 20 μl, isothermal amplification was carried out for 40 min at 63° C. until logarithmic phase was reached. The enzymatic reaction mixture solution consists of the following constituents: 0.3 mg of T4 Gene 32 protein (USB), 6 units of RNase inhibitor (Inrton), 0.5 unit of RNaseH (Epicentre), 20 units of Bst DNA polymerase (NEBM0275M), and 1 nM DNA-RNA-DNA hybrid probe. As a control of the example, a nucleic acid without the target nucleic acids was used.

### Example 6

### Detection of Gold Nanoprobe Aggregation

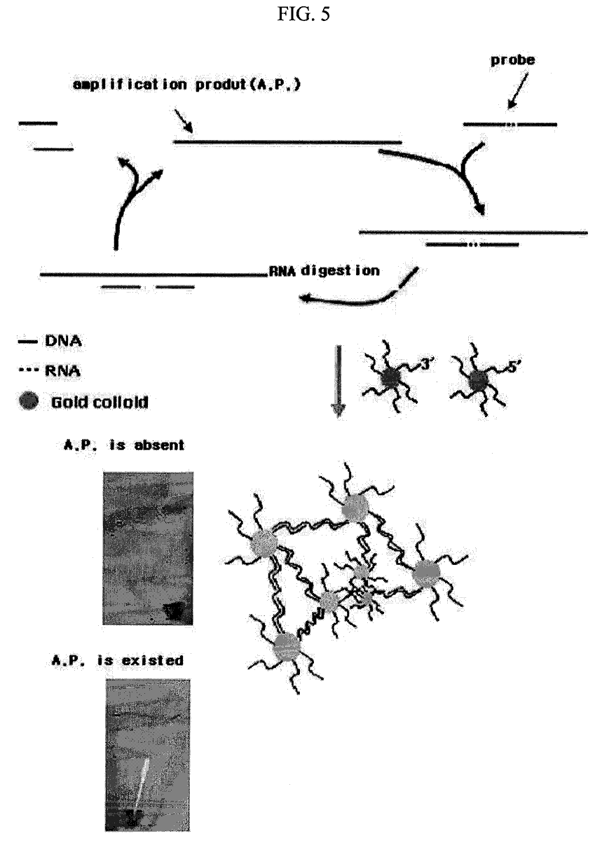

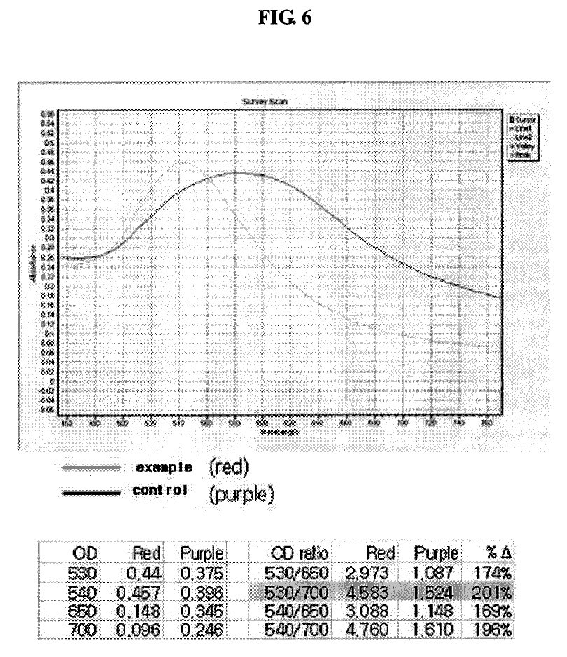

The probe (SEQ ID NOs: 5 and 6) labelled with gold nanoparticles prepared in Example 4 and 10 mM of phosphoric acid were added into 1 ml of amplification product obtained in Example 5, and then added with 0.5M NaCl to be a final reaction solution of 50 ml. The absorbance values at 530 nm and 700 nm were measured to calculate the ratio of OD₅₃₀/OD₇₀₀, and thus compared an experimental sample with a control sample. The larger the difference between the values is, the more effective it is (FIG. 5 and FIG. 6).

As shown in FIG. 5 and FIG. 6, it was confirmed that in experimental sample where a signal probe having a base sequence complementary to the gold nanooligoprobe existed, the gold nanoprobe was cross-linked by hybridization to induce gold nanoprobe aggregation, thus changing the maximum absorbance.

### Example 7

### Amplification of Target Nucleic Acids and a Signal Probe for HRP Detection

By referring to the whole base sequences of E. coli 0157:H7 (KCCM 40406), primers (SEQ ID NOs: 7˜10) and a probe (SEQ ID NO: 12) were prepared for selectively amplifying a specific gene region (stx-2).

| | |
|---|---|
| SEQ ID NO: 7: | 5′-CGTTCCGGAATGCAAATC-3′ |
| SEQ ID NO: 8: | 5′-CATCGTATACACAGGAGC-3′ |
| SEQ ID NO: 9: | 5′-TACCTTAAGGCATGGGCTGACTACTACTCACTGGTTTCATCATATCT-3′ |
| SEQ ID NO: 10: | 5′-CTGCTACGCGTGTGAAGATGACCCTGAACAGTGCCTGACGAAATTCT-3′ |
| SEQ ID NO: 12: | 5′-Fluorescein-TGTGACAGTGACAAAACGCAGAACT-GCT-Biotin-3′ |

The primers of SEQ ID NOs: 7 and 8 are external primers having a sequence complementary to Escherichia coli 0157:H7, and the primers of SEQ ID NOs: 9 and 10 are inner primers having a sequence complementary and a sequence non-specific to Escherichia coli 0157:H7. SEQ ID NO: 12 is a DNA/RNA/DNA hybrid probe for performing an amplification of a signal probe, in which the 3′ end thereof is labelled with a biotin and 5′ end thereof is labelled with a fluorescein. The underlined parts are RNA base sequences.

In order to anneal the primers with desired nucleic acids, a reaction mixture containing the external primers (SEQ ID NOs: 7 and 8), and inner primers (SEQ ID NOs: 9 and 10) was denatured for 5 min at 95° C., and cooled for 5 min at 63° C.

The reaction mixture consists of the following constituents: 5 mM Tris-HCl (pH 8.5), 10 mM of (NH₄)₂SO₄, 10 mM of MgSO₄, 4 mM of KCl, 0.5 mM of each dNTP (Fermentas), 2.9 mM of DTT, 0.1 mg of BSA, 10 mM of EGTA, 50 mM spermine, 0.08 mM of external primer, 0.126 mM of inner primer and 1 ng of E. coli 0157:H7 DNA.

Then, after adding enzymatic reaction mixture solution to the cooled reaction mixture to make a final volume of 20 μl, isothermal amplification was carried out for 40 min at 63° C. till logarithmic phase was reached. The enzymatic reaction mixture solution consists of the following constituents: 0.3 mg of T4 Gene 32 protein (USB), 6 units of RNase inhibitor (Inrton), 0.5 unit of RNaseH (Epicentre), 20 units of Bst DNA polymerase (NEBM0275M), and 1 nM DNA-RNA-DNA hybrid probe. As a control of the example, a nucleic acid without the target nucleic acids was used.

### Example 8

### Detection by HRP

170 ml of PBST binding buffer was added to amplification product obtained in Example 7 to prepare a reaction mixture consisting of the following constituents: 135 mM of NaCl, 2.7 mM of KCl, 8.1 mM of Na₂HPO₄, 1.5 mM KH₂PO₄, 0.05% Tween 20, 1/1000 diluted anti-F-HRP (Perkin Elmer, horseradish peroxidase conjugated anti-fluorescent antibody).

The reaction mixture was moved into streptavidin coating microplate well (Roche), and allowed to react for 10 min at 37° C. and 200 rpm. The supernatant in the well was removed and added with 300 ml of PBST washing buffer to wash, wherein the PBST washing buffer has the same composition as the above binding buffer except for the removal of an antibody. The washed well was added with 200 ml of HRP substrate, 3,3′,5,5′-tetramethylbenzidine (Bio-Rad, TMB), colordeveloped for 5 min in a dark place, and added with 100 ml of 1N H₂SO₄ to stop the reaction.

In order to determine the effectiveness of the samples and the control, the absorbance values at 465 nm were compared using the ELISA reader (Zenuth 340rt). It is determined that the larger the difference between the values is, the more effective it is.

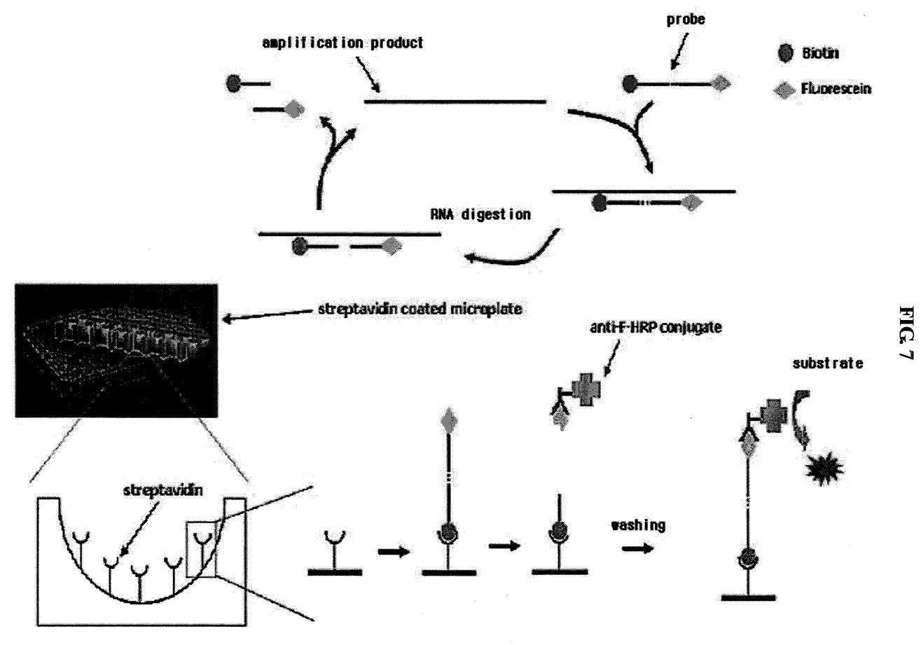

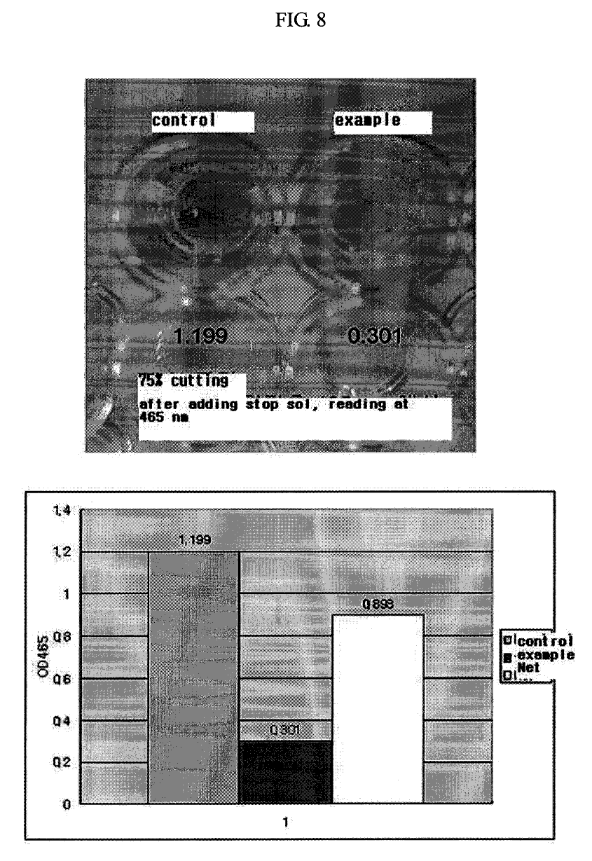

As a result, as shown in FIG. 7 and FIG. 8, it was confirmed that the fluorescence of the fluorescein-labelled probe was not colordeveloped by HRP (horse radish peroxidase) conjugated with anti-fluorescein in the experimental sample where amplification product existed.

### INDUSTRIAL APPLICABILITY

The present invention provides a method for amplifying target nucleic acids rapidly and exactly at isothermal temperature, and a method for detecting nucleic acids, which comprises simultaneously performing amplifications of nucleic acids and a signal probe. The method according to the present invention is convenient compared with the conventional methods, it is possible to amplify the target nucleic acids rapidly and exactly without a risk of contamination, and it can simultaneously amplify a signal probe, so that it can be applied to various genome project, such as detection and identification of a pathogen, detection of gene modification causing predetermined phenotype, detection of hereditary diseases or determination of sensibility to diseases, estimation of gene expression and apply to genome project, thus being useful for molecular biological studies and disease diagnosis.

What is claimed is:

1. A method for isothermal amplification of target nucleic acids, the method comprises:
(a) denaturing a reaction mixture containing (i) target nucleic acids, (ii) an external primer set having a base sequence complementary to the target nucleic acids, and (iii) RNA/DNA hybrid inner primer set having a base sequence partially complementary to the target nucleic acids; and
(b) amplifying said target nucleic acids at isothermal temperature after adding an enzymatic reaction mixture solution to the reaction mixture denatured in step (a), wherein the enzymatic reaction mixture solution contains RNase and DNA polymerase capable of strand displacement.

2. The method for isothermal amplification of target nucleic acids according to claim 1, wherein the external primer set is any one selected from the group consisting of: oligo DNA; oligo RNA; and hybrid oligo RNA/DNA.

3. The method for isothermal amplification of target nucleic acids according to claim 1, wherein RNA/DNA hybrid inner primer set is designed such that RNA region thereof has a base sequence non-complementary to the target nucleic acids and DNA region thereof has a base sequence complementary to the target nucleic acids.

4. The method for isothermal amplification of target nucleic acids according to claim 1, wherein the DNA polymerase is a thermostable DNA polymerase.

5. The method for isothermal amplification of target nucleic acids according to claim 4, wherein the thermostable DNA polymerase is any one selected from the group consisting of: Bst DNA polymerase; exo(−) vent DNA polymerase; exo(−) Deep vent DNA polymerase; exo(−) Pfu DNA polymerase; and Bca DNA polymerase.

6. The method for isothermal amplification of target nucleic acids according to claim 1, wherein the RNase is RNaseH.

7. The method for isothermal amplification of target nucleic acids according to claim 1, wherein the isothermal amplification is carried out at 50˜65° C.

8. A method for detecting nucleic acids, the method comprises:
(a) denaturing a reaction mixture containing (i) a nucleic acid sample for detecting target nucleic acids, (ii) an external primer set having a base sequence complementary to the target nucleic acids, and (iii) RNA/DNA hybrid inner primer set having a base sequence partially complementary to the target nucleic acids;
(b) simultaneously amplifying said target nucleic acids and said probe signals at isothermal temperature after adding an enzymatic reaction mixture solution to the reaction mixture denatured in step (a), wherein the enzymatic reaction mixture solution contains RNase and DNA polymerase capable of strand displacement, and DNA/RNA/DNA hybrid probe having a base sequence complementary to amplification products produced by the external primer and inner primer set; and
(c) detecting target nucleic acids using the amplified probe signals.

9. The method for detecting nucleic acids according to claim 8, wherein the external primer is any one selected from the group consisting of: oligo DNA, oligo RNA, and hybrid oligo RNA/DNA.

10. The method for detecting nucleic acids according to claim 8, wherein RNA/DNA hybrid inner primer set is designed such that RNA region thereof has a base sequence non-complementary to the target nucleic acids and DNA region thereof has a base sequence complementary to the target nucleic acids.

11. The method for detecting nucleic acids according to claim 8, wherein RNA/DNA hybrid inner primer set is designed such that the DNA/RNA/DNA hybrid probe consists of 25˜45 bases.

12. The method for detecting nucleic acids according to claim 11, wherein the DNA/RNA/DNA hybrid probe is designed such that the length of external DNA region thereof consists of 10˜20 bases and the length of inner RNA region thereof consists of 4˜6 bases.

13. The method for detecting nucleic acids according to claim 8, wherein the both end of DNA/RNA/DNA hybrid probe are labelled with markers.

14. The method for detecting nucleic acids according to claim 13, wherein the marker is selected from the group consisting of: biotin, fluorescent, dioxygenin, or dinitrophenyl.

15. The method for detecting nucleic acids according to claim 13, wherein the DNA/RNA/DNA hybrid probe has a phosphate group adhered to the 3′ end thereof.

16. The method for detecting nucleic acids according to claim 8, wherein the DNA polymerase is a thermostable DNA polymerase.

17. The method for detecting nucleic acids according to claim 16, wherein the thermostable DNA polymerase is any one selected from the group consisting of: Bst DNA polymerase, exo(−) vent DNA polymerase, and Bca DNA polymerase.

18. The method for detecting nucleic acids according to claim 8, wherein the RNase is RNaseH.

19. The method for detecting nucleic acids according to claim 8, wherein the amplification of the target nucleic acids and the probe signals is carried out at 50˜65° C.


SEQUENCE LISTING (12 sequences)

SEQ ID NO: 1 - DNA, 19 bp, Artificial Sequence
  outer primer
ggacgtcagaaaaccagaa

SEQ ID NO: 2 - DNA, 17 bp, Artificial Sequence
  outer primer
ggcagtgaagcccagat

SEQ ID NO: 3 - DNA, 41 bp, Artificial Sequence
  inner primer
uaagagaucgcccgucagccgctccgatcaccctcgcaaac

SEQ ID NO: 4 - DNA, 41 bp, Artificial Sequence
  inner primer
caacaugaccgacgcuugcccgcgccacgctccttaatctg

SEQ ID NO: 5 - DNA, 15 bp, Artificial Sequence
  probe
agtgacaaaacgcag

SEQ ID NO: 6 - DNA, 15 bp, Artificial Sequence
  probe
aactgctctggatgc

SEQ ID NO: 7 - DNA, 18 bp, Artificial Sequence
  outer primer
cgttccggaatgcaaatc

SEQ ID NO: 8 - DNA, 18 bp, Artificial Sequence
  outer primer
catcgtatacacaggagc

SEQ ID NO: 9 - DNA, 47 bp, Artificial Sequence
  inner primer
taccttaaggcatgggctgactactactcactggtttcatcatatct

SEQ ID NO: 10 - DNA, 47 bp, Artificial Sequence
  inner primer
ctgctacgcgtgtgaagatgaccctgaacagtgcctgacgaaattct

SEQ ID NO: 11 - DNA, 34 bp, Artificial Sequence
  probe
tgctgtgacagtgacaaaacgcagaactgctctg

SEQ ID NO: 12 - DNA, 28 bp, Artificial Sequence
  probe
tgtgacagtgacaaaacgcagaactgct